(19) United States
(12) Patent Application Publication
(10) Pub. No.: US 2003/0003552 A1
(43) Pub. Date: Jan. 2, 2003

(54) Method for kinetic resolution of racemates of alcohols or carboxylic acid esters

(76) Inventors: Fritz Theil, Berlin (DE); Helmut Sonnenschein, Leipzig (DE)

Correspondence Address: GUDRUN E. HUCKETT, LONSSTR. 53, WUPPERTAL, 42289, DE

(21) Appl. No.: 10/168,744

(22) PCT Filed: Dec. 18, 2000
(86) PCT No.: PCT/DE00/04536

(30) Foreign Application Priority Data
Dec. 19, 1999 (DE) 199 63 314.2
Dec. 19, 1999 (DE) 199 63 315.0
Feb. 8, 2000 (DE) 100 06 824.3

(51) Int. Cl. C12P 7/62; C12P 7/40

(52) U.S. Cl. 435/135; 435/136

(57) ABSTRACT

The invention relates to a method of kinetic resolution of racemates of alcohols or carboxylic acid esters. Fluorine phase marking of the quicker or more slowly reacting enantiomer is obtained by lipase-catalysed reaction of racemic alcohols with fluorinated carboxylic acid esters of racemic alcohols with water. The enantiomers are then extractively separated by division between an organic and a fluorine phase.

The present invention relates to a method for kinetic resolution of racemates of alcohols or carboxylic acid esters with one or several stereogenic centers. The invention is suitable in particular in the manufacture of pharmaceutical agents or plant protective agents.

The lipase-catalyzed kinetic resolution of racemates of alcohols by enantiomer-selective esterification or by enantiomer-selective hydrolysis of carboxylic acid esters of racemic alcohols is a well-established method in organic synthesis (F. Theil, Chem. Rev. 1995, 95, 2203-2227; U. T. Bornscheuer, R. J. Kazlauskas, Hydrolases in Organic Synthesis, Wiley-VCH, Weinheim, 1999). The slower-reacting enantiomer is retained as an alcohol or carboxylic acid ester while the faster reacting enantiomer is obtained as carboxylic acid ester or alcohol. Conventionally, alcohol and carboxylic acid ester are separated by chromatography (F. Theil et al., J. Org. Chem. 1994, 59, 388-393).

Simpler separations are obtained only when, for example, alcohol and ester differ with regard to their solubility properties to such an extent that an extractive separation between aqueous and organic phase is possible, a method which can be realized however only in exceptional situations (P. Stead et al., Tetrahedron: Asymmetry 1996, 7, 2247-2250).

A further possibility for the separation of ester and alcohol is provided when the formed ester is acidic, which can be realized, for example, by esterification with cyclic carboxylic acid anhydrides. However, this is also not a generally applicable method because cyclic anhydrides do not constitute optimal acyl donors for lipase-catalyzed esterifications. Moreover, acidic compounds reduce the lipase activity (B. Berger et al. Tetrahedron: Asymmetry 1990 1, 541-546; U. T. Bornscheuer, R. J. Kazlauskas, Hydrolases in Organic Synthesis, Wiley-VCH, pp. 44-47, Weinheim, 1999).

Racemic carboxylic acid esters with one or several stereogenic centers in the carboxyl group of the molecule can be esterified by lipase-catalyzed alcoholysis with an alcohol which is different from the one already present within the ester molecule. The two resulting enantiomeric carboxylic acid esters are however separable only by means of complex chromatographic methods so that this procedure is without practical importance. Conventionally, the enantiomeric separation is realized by lipase-catalyzed hydrolysis (U. T. Bornscheuer, R. J. Kazlauskas, Hydrolases in Organic Synthesis, Wiley-VCH, Weinheim, 1999).

It is therefore an object of the invention to provide an improved method for resolution of racemic alcohols or carboxylic acid esters.

According to the invention, this object is solved by a method for kinetic resolution of racemates of alcohols or carboxylic acid esters with one or several stereogenic centers, in which by lipase-catalyzed reaction of racemic alcohols with fluorinated acylation agents or of esters of racemic carboxylic acids with fluorinated alcohols or of fluorinated carboxylic acid esters of racemic alcohols with water a fluorine phase marking of the faster or slower reacting enantiomer is possible. The enantiomers are subsequently extractively separated by distribution between the organic and fluorine-containing phases.

In this connection, fluorine-containing solvents are to be understood as solvents with a high degree of fluorination which cannot be admixed with conventional organic solvents.

According to the invention, racemic alcohols with one or several stereogenic centers are acylated enantiomer-selectively by lipase catalysis with per-fluorinated acylation agents of the formula I

R—(CH₂)ₙ—COOR¹ (I)

in which

R can be a per-fluorinated alkyl group such as —(CF₂)ₘ—CF₃,

wherein m is an integer from 3 to 18,

or

a per-fluorinated aromatic group such as C₆F₄X

and

X is fluorine or a per-fluorinated alkyl group,

R¹ is alkyl, vinyl, aryl, 2-cyanoethyl, 2,2,2-trifluoroethyl or 2,2,2-trichloroethyl

and

n can be an integer from 1 to 4.

According to the invention, as shown in the Schematic 1, in the presence of a lipase a per-fluorinated group is transferred onto the faster reacting enantiomer so that this enantiomer becomes soluble in the fluorine-containing phase and is extractively separated from the organic phase in which the unreacted enantiomer is retained.

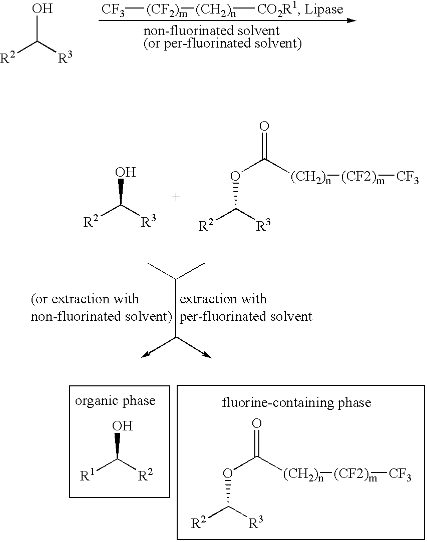

Schematic 1:

R² and R³=alkyl, alkenyl or aryl

R¹, n and m as defined above

According to the invention, racemic carboxylic acid esters with one or several stereogenic centers in the acyl group are subjected to a lipase-catalyzed alcoholysis with a per-fluorinated alcohol of the formula II

CF₃—(CF₂)ₘ—(CH₂)ₙ—OH (II)

in which

m is an integer of 4 to 18 and n is either 0 or an integer from 1 to 4.

According to the invention, as shown in the Schematic 2, in the presence of a lipase a per-fluorinated group is transferred onto the faster reacting enantiomer so that this enantiomer becomes soluble in the fluorine-containing phase and can be extractively separated from the organic phase in which the unreacted enantiomer is retained.

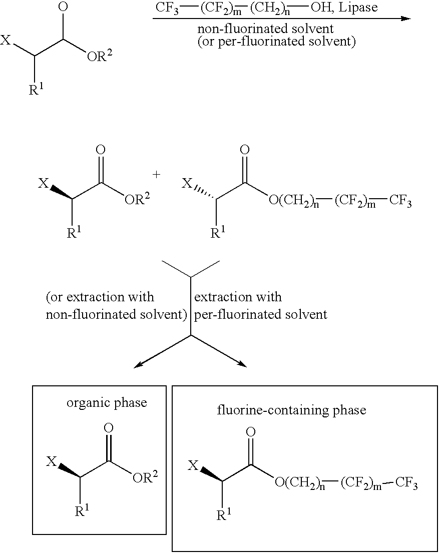

Schematic 2:

R¹ and R²=alkyl, vinyl or aryl

X=alkyl, alkenyl, alkoxy, aryl, aryloxy, or halogen

According to the invention, the reactions are carried out by means of a lipase of microbial, plant-based or animal-based origin at room temperature or elevated temperature either in solvents conventionally employed for such reactions, such as aliphatic and aromatic hydrocarbons, ethers, tertiary alcohols or also chloro hydrocarbons. Subsequently, the per-fluorinated enantiomer is extracted with a per-fluorinated solvent which is immiscible with the non-fluorinated organic solvent. As an alternative, the lipase-catalyzed reactions are carried out in a per-fluorinated solvent and, subsequently, the non-fluorinated enantiomer is extracted by means of non-fluorinated organic solvents.

According to the invention, the lipase-catalyzed kinetic resolution of racemates can also be performed in a two-phase system of organic and fluorine-containing solvents at room temperature or low temperature. The phase homogenization during the chemical reaction is realized either by heating or controlled, gentle exposure to microwaves. The phase separation and the correlated product separation is achieved by cooling of the reaction mixture below the phase mixing temperature. In this way, the separation of the enantiomers can be performed with good yields.

According to the invention, racemic alcohols with one or several stereogenic centers are converted with per-fluorinated acylation agents into an ester of the formula III

R—(CH₂)ₙ—COOCHR¹R² (III)

in which

R is a per-fluorinated alkyl group such as —(CF₂)ₘ—CF₃,

wherein m can be an integer from 3 to 18,

or

a per-fluorinated aromatic group such as C₆F₄X

and

X is fluorine or a per-fluorinated alkyl group,

R¹, R² are alkyl, alkenyl, aryl, or heteroaryl

and

n can be an integer from 0 to 4,

and subsequently, as is known in the art, are hydrolyzed with ester-cleaving enzymes, preferably lipases, in an enantiomer-selective way.

According to the invention, as shown in Schematic 3,

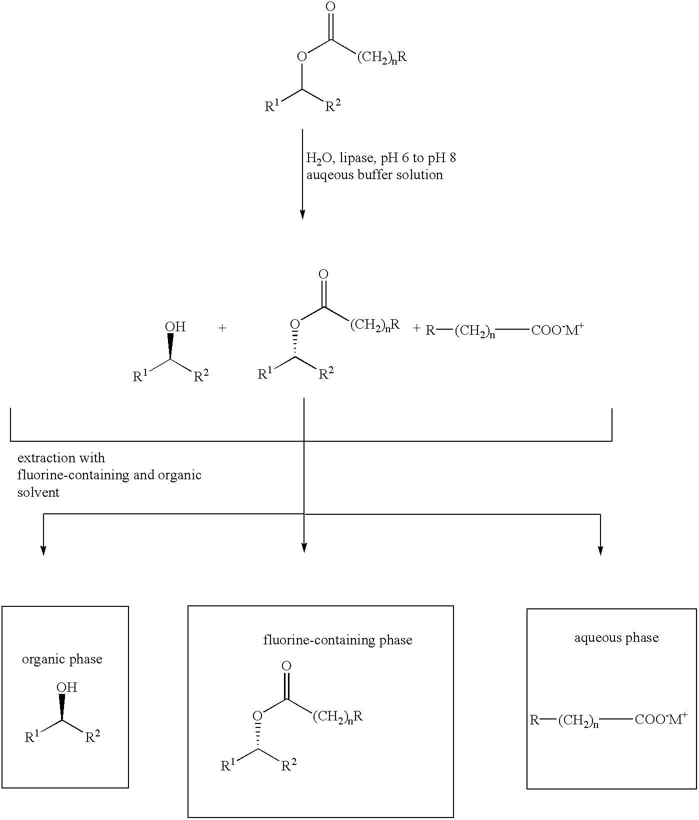

Schematic 3

R, R¹, R² and n as defined above

M⁺ alkali cation such as Na⁺ or K⁺

in the presence of an ester-cleaving enzyme, preferably, a lipase, the per-fluorinated group is cleaved off the faster-reacting enantiomer so that this enantiomer is no longer soluble in fluorine-containing solvents. According to the invention, in the subsequent distribution of the reaction products between organic, fluorine-containing, and aqueous phase, the free alcohol is in the organic phase, the uncleaved ester in the fluorine-containing phase, and the salt of the carboxylic acid in the aqueous phase.

According to the invention, the hydrolysis is carried out with a lipase of microbial, plant or animal origin either in solvents conventionally employed for such reactions, such as aqueous buffer solution or also with addition of a solvent miscible with water or immiscible with water in homogenous or heterogeneous phase. The pH value in the aqueous phase is maintained at constant values between 6 and 8, preferably at a pH of 7, by addition of suitable bases, such as aqueous sodium or potassium hydroxide solution, as is conventional in the art. Subsequently, an organic solvent which is immiscible with water and a fluorine-containing solvent are added to the reaction mixture so that a three-phase system results. As an alternative to this procedure, the reaction mixture is extracted with a suitable organic solvent which is immiscible with water and the organic solvent is subsequently evaporated. The resulting residue is distributed between suitable organic and fluorine-containing solvents. After extractive separation, the released alcohol is isolated from the organic phase and the unreacted carboxylic acid ester is isolated from the fluorine-containing phase. The alkali salt of the released carboxylic acid remains in the aqueous phase. In this way the separation of the enantiomers can be performed with good yields.

With the aid of the following examples the invention will be explained in more detail.

### EXAMPLE 1

To a mixture of racemic 1-phenyl ethanol (0.366 g, 3 mmol) and acetonitrile (10 ml), 4,4,5,5,6,6,7,7,8,8,9,9,10,10,11,11,11-heptadecafluoro undecane acid-2,2,2-trifluoro ethyl ester (2.58 g, 4.5 mmol) and Candida antarctica B lipase (0.1 g) are added, and the mixture then stirred at room temperature until 50% of the alcohol has reacted. The enzyme is filtered off and the filtrate is concentrated in vacuum to a dryness. The residue is taken up in methanol (10 ml) and the mixture is extracted five times with n-perfluorohexane. The organic phase contains (S)-1-phenyl ethanol with an enantiomeric excess greater 95%. The fluorine-containing phase contains (R)-4,4,5,5,6,6,7,7,8,8,9,9,10,10,11,11,11-heptadecafluoro undecane acid-1-phenyl ethyl ester with an enantiomeric excess of greater 95%.

### EXAMPLE 2

To a mixture of racemic 1-phenyl ethanol (0.366 g, 3 mmol) and n-perfluorohexane (10 ml), 4,4,5,5,6,6,7,7,8,8,9,9,10,10,11,11,11-heptadecafluoro undecane acid-2,2,2-trifluoroethyl ester (2.58 g, 4.5 mmol) and Candida antarctica B lipase (0.1 g) are added, and the mixture shaken at room temperature until 50% of the alcohol has reacted. The enzyme is filtered off and the filtrate is extracted five times with methanol. The organic phase contains (S)-1-phenyl ethanol with an enantiomeric excess of greater 95%. The fluorine-containing phase contains (R)-4,4,5,5,6,6,7,7,8,8,9,9,10,10,11,11,11-heptadecafluoro undecane acid-1-phenyl ethyl ester with an enantiomeric excess of greater 95%.

### EXAMPLE 3

To a mixture of racemic 1-phenylethanol (0.366 g, 3 mmol), acetonitrile (5 ml) and n-perfluorohexane (5 ml), 4,4,5,5,6,6,7,7,8,8,9,9,10,11,11,11-heptadecafluoro undecane acid-2,2,2-trifluoroethyl ester (2.58 g, 4.5 mmol) and Candida antarctica B lipase (0.1 g) are added, and the mixture stirred at 60° C. until 50% of the alcohol has reacted. The enzyme is filtered off and the filtrate is cooled to room temperature. Subsequently, the phases are separated. The organic phase contains enriched (S)-1-phenyl ethanol. The fluorine-containing phase contains enriched (R)4,4,5,5,6,6,7,7,8,8,9,9,10,10,11,11,11-heptadecafluoro undecane acid-1-phenyl ethyl ester.

### EXAMPLE 4

To a mixture of racemic 2-phenyl butyric acid vinyl ester (0.950 g, 5 mmol), 1H,1H,2H,2H-perfluorodecane-1-ol (1.4 g, 3 mmol) and tertiary butyl methyl ether (10 ml), Candida antartica B lipase (0.1 g) is added, and the mixture stirred at room temperature until 30% of the vinyl ester has reacted. The enzyme is filtered off and the filtrate is concentrated in vacuum to dryness. The residue is taken up in methanol (10 ml) and the mixture is extracted five times with n-perfluorooctane. The organic phase contains an enantiomer of the vinyl ester in an enriched form. The fluorine-containing phase contains 2-phenyl butyric acid-3,3,4,4,5,5,6,6,7,7,8,8,9,9,10,10,10-heptadecafluoro decyl ester with enantiomeric excess of 80%.

### EXAMPLE 5

To a mixture of racemic 2-phenyl butyric acid vinyl ester and (0.950 g, 5 mmol), 1H,1H,2H,2H-perfluorodecane-1-ol (1.4 g, 3 mmol) and n-perfluorooctane (10 ml), Lipozyme IM 20 (lipase of Mucor miehei) (0.250 g) is added, and the mixture shaken at room temperature until 30% of the vinyl ester has reacted. The enzyme is filtered off and the filtrate is extracted five times with methanol. The organic phase contains an enantiomer of the vinyl ester in an enriched form. The fluorine-containing phase contains 2-phenyl butyric acid-3,3,4,4,5,5,6,6,7,7,8,8,9,9,10,10,10-heptadecafluoro decyl ester with an enantiomeric excess of greater 90%.

### EXAMPLE 6

To a mixture of racemic 2-phenyl butyric acid vinyl ester (0.950 g, 5 mmol), 1H,1H,2H,2H-perfluorodecane-1-ol, (1.4 g, 3 mmol), n-perfluorooctane (3 ml), and acetonitrile (7 ml), Lipozyme IM 20 (lipase of Mucor miehei) (0.250 g) is added, and the mixture stirred at 60° C. until 30% of the vinyl ester has reacted. The enzyme is filtered off and the filtrate is cooled to room temperature. The resulting phases are separated, wherein the organic phase contains an enantiomer of the vinyl ester in an enriched form. The fluorine-containing phase contains 2-phenyl butyric acid-3,3,4,4,5,5,6,6,7,7,8,8,9,9,10,10,10-heptadecafluoro decyl ester in an enantiomer-enriched form.

### EXAMPLE 7

To a mixture of racemic 4,4,5,5,6,6,7,7,8,8,9,9,10,10,11,11,11-heptadecafluoro undecane acid-1-phenyl ethyl ester (1.79 g, 3 mmol) in an aqueous phosphate buffer solution of a pH of 7.0 (20 ml) and acetonitrile (5 ml), Candida antarctica B lipase (0.1 g) is added, and the mixture stirred while maintaining a constant pH value (continuous titration with 0.1 N sodium hydroxide solution) at 37° C. until 50% of the ester has been hydrolyzed. The enzyme is filtered off and the filtrate is extracted with ethyl acetate. The combined organic extracts are then concentrated in vacuum to dryness. The residue is taken up in methanol (10 ml) and the resulting mixture is extracted five times with n-perfluorohexane. The organic phase contains (R)-1-phenyl ethanol with an enantiomeric excess of greater 95%. The fluorine-containing phase contains (S)-4,4,5,5,6,6,7,7,8,8,9,9,10,10,11,11,11-heptadecafluoro undecane acid-1-phenyl ethyl ester within an enantiomeric excess of greater 95%.

1. A method for kinetic resolution of racemates of alcohols or carboxylic acid esters with one or several stereogenic centers, characterized in that by lipase-catalyzed reaction of racemic alcohols with fluorinated acylation agents or of fluorinated carboxylic acid esters of racemic alcohols with water or of esters of racemic carboxylic acids with fluorinated alcohols the fluorine phase marking of the faster or slower reacting enantiomer is achieved and in that the enantiomers are subsequently separated by distribution between organic phase and fluorine-containing phase.

2. Method according to claim 1, characterized in that the alcohols with one or several stereogenic centers are acylated enantiomer-selectively by lipase-catalyzed esterification in organic or fluorine-containing solvents with fluorinated acylation agents of the formula I
R—(CH₂)ₙ—COOR¹ (I)
in which
R is a per-fluorinated alkyl group such as —(CF₂)ₘ—CF₃,
wherein m can be an integer from 3 to 18
or
a per-fluorinated aromatic group such as C₆F₄X
and
X is fluorine or a per-fluorinated alkyl group,
R¹ is alkyl, vinyl, aryl, 2-cyanoethyl, 2,2,2-trifluoroethyl or 2,2,2-trichloroethyl
and
n can be an integer from 1 to 4,
and in that the obtained enantiomers are separated from one another by extractive distribution between organic phase and fluorine-containing phase.

3. Method according to claim 1, characterized in that the carboxylic acid esters with one or several stereogenic centers in the acyl group are reacted by lipase-catalyzed alcoholysis in organic or fluorine-containing solvents with fluorinated alcohols of the formula II
CF₃—(CF₂)ₘ—(CH₂)ₙ—OH (II)
in which
m is an integer of 4 to 18 and n is either 0 or an integer from 1 to 4,
and in that the obtained enantiomers are separated from one another by extractive distribution between organic phase and fluorine-containing phase.

4. Method according to claim 2 or 3,characterized in that the lipase-catalyzed enantiomer-selective acylation or alcoholysis is carried out in a two-phase system of organic phase and fluorine-containing phase, wherein the phase homogenization during the reaction is realized by heating or controlled exposure to microwaves, and subsequently the phase separation and thus the product separation is carried out by cooling the reaction mixture below the phase mixing temperature.

5. Method according to claim 2 or 3, characterized in that acylation or alcoholysis is carried out in aliphatic or aromatic hydrocarbons, ethers, tertiary alcohols or chlorohydrocarbons and in that the extraction of the fluorinated enantiomer is realized with a perfluorinated solvent.

6. Method according to claim 2 or 3, characterized in that the acylation or alcoholysis is performed in a per-fluorinated solvent and in that the extraction is carried out with a non-fluorinated solvent.

7. Method according to claim 1, characterized in that for kinetic resolution of racemates of fluorinated carboxylic acid esters of racemic alcohols with one or several stereogenic centers the corresponding racemic alcohols are converted with fluorinated acylation agents to their esters of the formula III
R—(CH₂)ₙ—COOCHR¹R² (III)
in which
R is a per-fluorinated alkyl group such as —(CF₂)ₘ—CF₃,
wherein m can be an integer from 3 to 18,
or
a per-fluorinated aromatic group such as C₆F₄X
and
X is fluorine or a per-fluorinated alkyl group,
R¹, R² are alkyl, alkenyl, aryl, or heteroaryl
and
n can be an integer from 0 to 4,
wherein these esters are subsequently hydrolyzed by enzyme catalysis enantiomer-selectively and the obtained enantiomers are separated from one another by extractive distribution between organic phase and fluorine-containing phase.

8. Method according to claim 7, characterized in that the hydrolysis is carried out in aqueous buffer solution.

9. Method according to claim 7 or 8, characterized in that the hydrolysis is carried out at a pH value of 6 to 8, preferably 7.

10. Method according to one of the claims 7 to 9, characterized in that the hydrolysis is carried out in aqueous buffer solution in the presence of an organic solvent which is miscible or immiscible with water.

11. Method according to one of the claims 1 to 10, characterized in that the lipase-catalyzed esterification is carried out at room temperature or a higher temperature.

12. Method according to one of the claims 1 to 11, characterized in that the lipase is of microbial, plant or animal origin.
